(19) United States
(12) Patent Application Publication
(10) Pub. No.: US 2019/0267994 A1
(43) Pub. Date: Aug. 29, 2019

(54) PATIENT CONTROLLED ELECTRICAL UTILIZATION RECEPTACLE

(71) Applicant: Michael Galbierz, St. Louis, MO (US)

(72) Inventor: Michael Galbierz, St. Louis, MO (US)

(21) Appl. No.: 16/283,434

(22) Filed: Feb. 22, 2019

Related U.S. Application Data
(60) Provisional application No. 62/636,467, filed on Feb. 28, 2018.

(51) Int. Cl. H03K 17/96; A61M 16/00

(52) U.S. Cl. CPC H03K 17/962; G08B 5/36; A61M 16/022

(57) ABSTRACT

A patient controlled utilization receptacle comprises a control circuit, a switched circuit. The control circuit includes a touch control switch, a control wire extending from the touch control switch, and an input to a relay. The switched circuit includes an output from the relay and a switched outlet. The patient, by contacting the control wire, will cause activate the touch control switch. The touch control switch will then send a signal to the relay to close the switched circuit to provide power to the outlet. The patient can thus activate equipment plugged into the switched outlet without help from an aide.

### CROSS-REFERENCE TO RELATED APPLICATIONS

This application claims priority to U.S. App. No. 62/636,467 filed Feb. 28, 2018, entitled “Patient Controlled Electrical Utilization Receptacle”; said application being incorporated herein by reference.

### STATEMENT REGARDING FEDERALLY SPONSORED RESEARCH OR DEVELOPMENT

Not Applicable.

### BACKGROUND

This application relates to a switched electrical utilization receptacle, and in particular, to a utilization receptacle that can be controlled (i.e., activated and deactivated) by a quadriplegic patient.

Quadriplegic patients are not able to move their arms or legs, and have virtually no motion. Because of this, they need an extensive amount of care that they are not able to provide for themselves. As an example, a quadriplegic patient who is not able to swallow will need accumulated saliva suctioned from his/her mouth. This requires virtually constant monitoring by a care giver to activate a suction device on a regular, and frequent, basis, or they aspirate and may die.

It would be desirable to be able to provide quadriplegic patients who have the ability to move a finger, their head, or even just their lips, chin, or tongue the ability to activate such devices on their own, thereby relieving the care giver of the constant vigil and giving the patient a sense of independence in their care.

### BRIEF SUMMARY

Briefly stated, a patient controlled utilization receptacle comprises, a touch control switch which is responsive to changes in capacitance, a control wire connected to an input of the touch control switch and which is of a sufficient length to be positionable adjacent the patient to be touched by the patient, a relay having input contacts electrically connected to an output of the touch control switch, and at least one contact plate movable between an on position in which the contact plate connects a pair of load contacts and an off position in which the contact plate is disengaged from the pair of load contacts; and a patient-controlled utilization outlet into which equipment, to be activated, can be plugged. The patient-controlled utilization outlet is electrically connected to the load contacts of the rely switch.

The touch control switch, control wire, said input to said relay define a control circuit of the patient controlled utilization receptacle, and the contact plate and load contacts of the relay and the patient-controlled utilization outlet define a switched circuit of said patient controlled utilization receptacle. Activation of the touch control switch activates the relay to move the contact plate from the off position to the on position, to thereby close the switched circuit to provide power to the patient-controlled utilization outlet.

According to an aspect of the device, the contact plate is moved from the “on” position to the “off” position to thereby open the switched circuit by a second activation of the touch control switch; such that the patient controls both activation and deactivation of equipment plugged into the patient-controlled utilization outlet.

According to an aspect of the device, the device can further include a by-pass circuit containing a by-pass outlet; such that said by-pass outlet is continuously powered and is not affected by operation of the touch control switch circuit.

According to an aspect of the device, the by-pass outlet and the patient-controlled utilization outlet can be proximate each other on said patient controlled utilization receptacle. However, the two outlets are electrically isolated from each other.

According to an aspect of the device, the device can include an indicator light in the switched circuit. The indicator light is when the switched circuit is closed to indicate that power is flowing to the patient-controlled utilization outlet.

According to an aspect of the device, the device can include a fuse or a current transformer circuit in the control wire or between the control wire and the touch control switch.

According to an aspect of the device, the device the circuits of the patient controlled utilization receptacle are contained within an enclosure, and the control wire extends through a panel of the enclosure; whereby if the enclosure is made from metal, the control wire is electrically insulated from said control panel.

According to an aspect of the device, the device the load contacts of the relay are normally open contacts.

According to an aspect of the device, the device the relay is a double-pole double-through relay comprising first and second pairs of load contacts and the patient controlled utilization outlet is connected to the first pair of load contacts and the indicator light.

### BRIEF DESCRIPTION OF THE DRAWINGS

FIG. 1 is an electrical schematic for a patient controlled electrical utilization receptacle;

FIGS. 2A and 2B are perspective views of the patient controlled electrical utilization receptacle; and

FIG. 3 is a photograph of a patient with a suction pump plugged into the patient controlled electrical utilization receptacle.

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what I presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

Referring to the drawings, a patient controlled electrical utilization receptacle 10 includes an utilization outlet 12, a touch control switch 14, and a solenoid operated double-pole, double-throw (DPDT) relay 40 which are part of a circuit 18. The touch control switch 14 can include a capacitive responsive switching circuit as described in U.S. Pat. Nos. 4,668,876 and 5,796,183, which are incorporated herein by reference. As is known, such switches allow for activation of a controlled device by touching a conductive portion of the device. Importantly, such switches are zero force switches and can be activated without the need to apply any force. That is, the switch is controlled merely by touch.

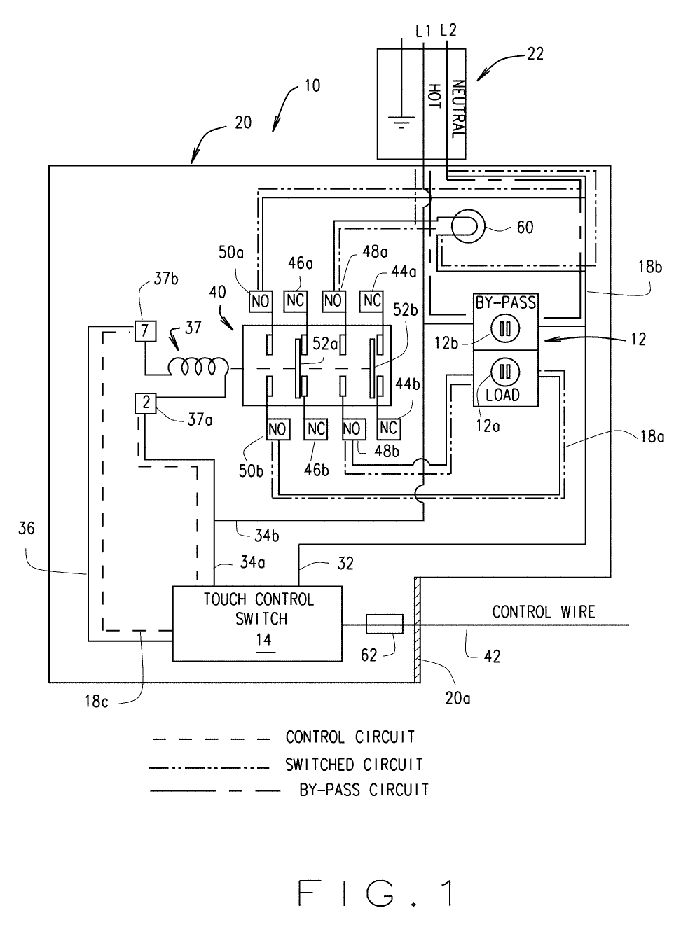

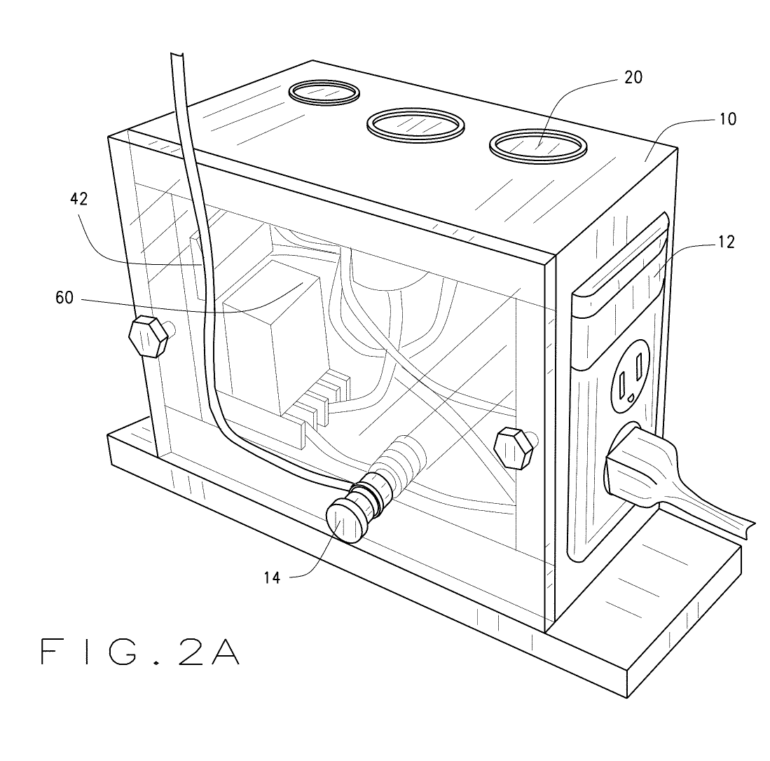

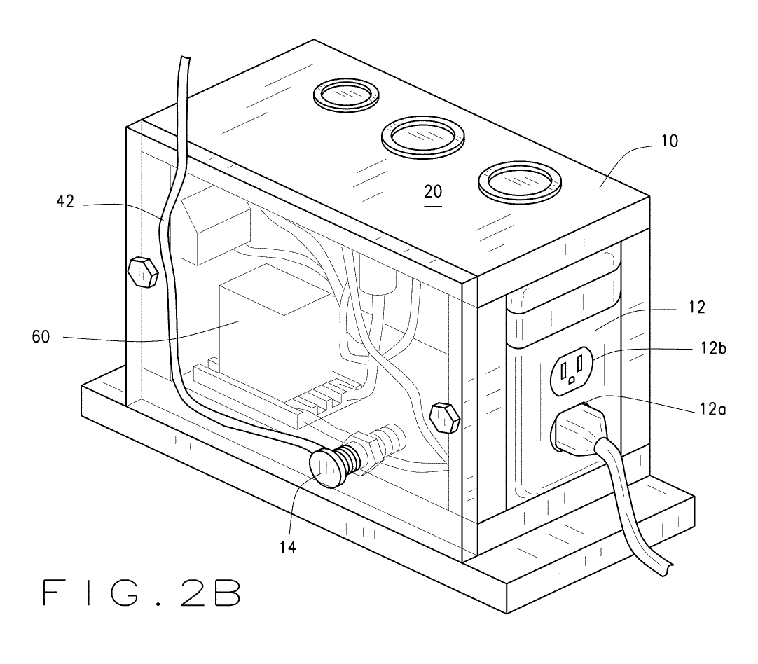

The circuit 18 is contained in a housing or enclosure 20. A power cord (including a plug) 22 extends from the enclosure to connect the utilization receptacle 10 to a source of power (such as by plugging the power cord into an electrical wall outlet). The utilization outlet device 12 comprises two isolated outlets 12a,b (at least one of which is a patient controlled utilization outlet) which are accessible externally of the enclosure 20 so that the power cord PC of equipment or a load E, such as a suction pump, can be plugged into the patient controlled utilization outlet to be controlled (i.e., at least activated) by the patient. As will be described below, the circuit 18 includes a switched circuit 18a which includes the switched (or patient controlled) utilization outlet 12a such that the equipment/load E can be activated and deactivated by means of the touch control switch 14, a by-pass circuit 18b which includes the utilization outlet 12b, such that the utilization outlet 12b is continuously powered, and a control circuit 18c, which as described below, will open and close the switched circuit 18a. The three circuits are shown in FIG. 1 by means of tracing lines adjacent the wires of the circuit.

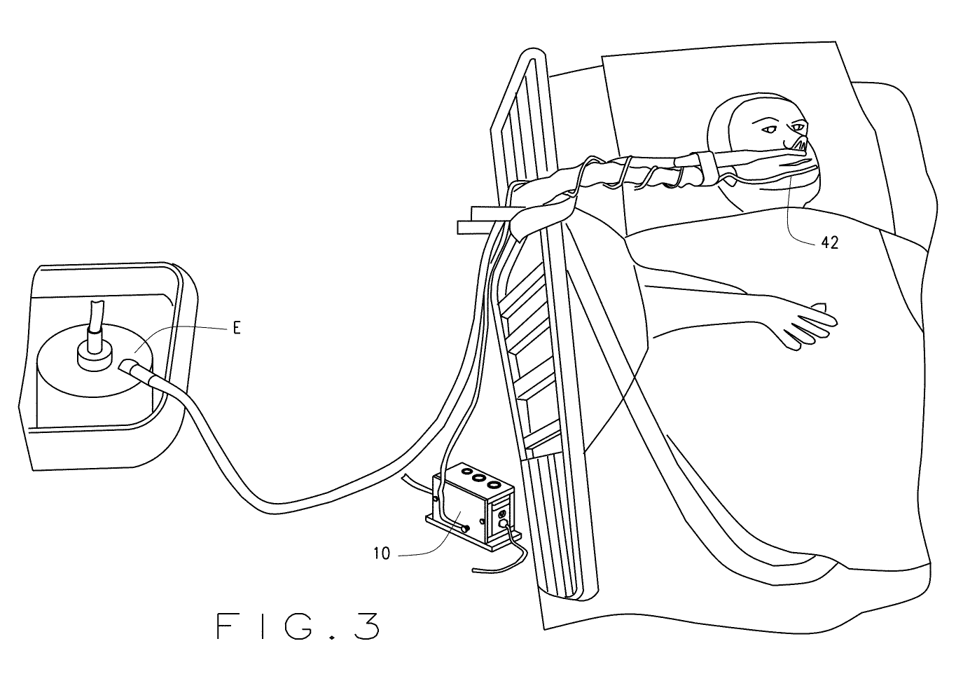

Turning to FIG. 1, a hot (black) wire 32 electrically connects the touch control switch 14 to neutral wire L2 of the plug 22. Two control (red and white) wires 34a, 36 electrically connect outputs from the touch control switch 14 to the opposite contacts 37a,b of a coil 37 for the solenoid of the double-pole, double-throw relay 40. The red wire 34a is electrically connected to the hot or live wire L1 of the plug 22 via a wire 34b. Lastly, a control wire 42 extends from the control switch through a portion 20a of the enclosure 20 in a manner in which the control wire is electrically insulated from the housing. The housing portion 20a can be non-inductive. As seen in FIG. 3, the control wire 42 is positioned adjacent a patient such that the patient can selectively touch the control wire 42, for example, with his chin, lip, tongue, etc. The end of the control wire can be positioned close to any body part the patient can move (such as a finger, lips, chin, tongue, etc.). By touching the control wire 42, the patient can activate the touch control switch 14 to control a device plugged into the patient-controlled utilization outlet 12a. As noted above, the touch control switch 14 is responsive to changes in capacitance, and thus does not require that the patient exert any force to activate the control switch 14.

Returning to FIG. 1, the relay 40 includes two pair of normally closed (NC) contacts 44a,b and 46a,b and two pair of normally open (NO) contacts 48a,b and 50a,b. Additionally, the relay 40 includes a pair of contact plates 52a,b which move between an “on” and an “off” position when the coil 37 is activated and deactivated, respectively. In the “on” position, the contact plates 52a,b extend across, and electrically connect, the normally open contacts 48a,b and 50a,b, respectively. In the “off” position (shown in FIG. 1), the contact plates 52a,b extend across, and electrically connect, the normally closed contacts 44a,b and 46a,b, respectively. The normally open contacts 48b and 50b are electrically connected to the opposite poles of the switched utilization outlet 12a. The normally open contact 50a is electrically connected to the neutral wire L2 of the plug 22; and the normally open contact 48a is electrically connected to the live or hot wire L1 of the plug 22. As can be appreciated, when the coil is activated to move the contact plates 52a,b to electrically connect the contacts 50a,b and 48a,b, the switched circuit will be closed, and power will flow through to the switched utilization outlet 12a. A light 60, such as an LED, is electrically connected between the switch contact 48a and a wire electrically connected to the neutral wire L2 of the plug, such that the light 60 will be activated when the relay 40 is in the “on” position.

The by-pass circuit includes the outlet 12b of the utilization outlet device 12, as noted above, which is electrically connected on opposite poles to the live wire L1 and neutral wire L2 of the plug 22. The by-pass outlet 12b is thus powered whenever the patient activated electrical utilization receptacle 10 is plugged into a wall outlet.

In operation, the equipment/load E (such as a suction pump) will be plugged into the patient-controlled utilization outlet 12a of the patient activated utilization receptacle 10 and the control wire 42 will be positioned such that the patient can make contact the control wire 42 on his/her own when it is necessary to activate the equipment/load E. When the patient needs to activate the equipment/load E, the patient touches the control wire 42. The change in capacitance sensed by the touch control switch 14 will cause the touch control switch 14 to send a signal to the relay 40 to activate the coil 37, which will, in turn, move the contact plates 52a,b to their “on” position, thereby closing the switched circuit 18a, sending power to the patient-controlled utilization outlet 12a, and activating the equipment/load E. When use of the equipment is no longer needed, the patient can again make contact with the control wire, which will cause the touch control switch 14 to send a signal to the coil to move the contact plates 52a,b to their “off” position, thereby opening the switched circuit 18a, and deactivating the equipment/load. The relay 40 could be a timed on/off delay relay. Such a relay would automatically function to open the switched circuit 18a after a predetermined period of time. Thus, the patient would not need to positively activate the relay a second time to turn off the equipment. Rather, the relay would automatically open the circuit after a determined time period to turn off the equipment.

FIG. 1 shows a fuse 62 in the control wire 42 to prevent an undue amount of electrical current from reaching the patient. The fuse can, for example, be a 5 milliamp fuse. A current transformer circuit could be used in place of the fuse to accomplish the same function. As noted above, the wall panel 20a, through which the control wire 42 extends is electrically insulated from the control wire. Preferably, the panel 20a is also non-inductive. This isolates that control wire from the remainder of the enclosure 20, so that the touch control switch 14 will not be inadvertently operated by someone who touches the enclosure. The electrical isolation of the housing panel 20a from the control wire, and the non-inductive property of the panel 20a will also help prevent any contact between the control wire and the enclosure from affecting the operation of the touch control switch 14 (and hence of the switched circuit). The concern or need to electrically insulate the control wire from the enclosure exists if the enclosure is made from a conductive metal. If the enclosure were made from a plastic, or other non-conductive (and non-inductive) materials the concerns regarding inadvertent activation or deactivation of the equipment plugged into the patient-controlled switch by inadvertent contact with the enclosure is of less concern. If desired, all the hardware in the enclosure 20 could be encapsulated and/or insulated.

It is noted that the patient activated utilization receptacle 10 is not provided with an on/off switch. This will avoid the possibility of a care giver setting up the patient activated utilization receptacle 10 and forgetting to activate (turn on) the patient activated utilization receptacle, or of an on/off switch being inadvertently set to an “off” position. Rather, it is preferred that the patient activated utilization receptacle be activated upon being plugged into the wall, so that no additional steps are necessary for the patient to be able to activate and deactivate the equipment E. However, an on/off switch could be provided, if desired.

As noted, the patient controlled electrical utilization receptacle is disclosed for use with a pump, and in particular a vacuum pump, which will allow the patient to control suctioning of saliva from his/her mouth without the need to call for assistance. In the hospital, the vacuum systems run continuously and nurses must shut it off to eliminate the suction and noise. With the disclosed patient controlled electrical utilization receptacle there the vacuum system need not run continuously, which will serve to reduce some of the noise in a patient room.

As various changes could be made in the above constructions without departing from the scope of the invention, it is intended that all matter contained in the above description or shown in the accompanying drawings shall be interpreted as illustrative and not in a limiting sense. For example, it may be desirable to have both utilization outlets 12a,b be patient activated, in which case, the utilization outlets 12a,b would not be electrically isolated from each other. The relay is a double-pole double-throw relay which allows for the indicator light 60 to be electrically connected to a different pair of contacts from the patient controlled utilization outlet 12a. The relay could be a single-pole switch in which case the indicator light would be connected either to the same contacts of the relay as the patient-controlled utilization outlet 12a or electrically connected to the poles of the patient controlled utilization outlet 12a. Although the patient activated utilization receptacle 10 is described to allow a patient to activate medical equipment, such as a suction pump, it will be apparent that virtually any electrical product can be turned on and off using the patient activated utilization receptacle 10. This includes, for example, other medical equipment, entertainment devices (such as TVs), call signals, etc. These examples are merely illustrative.

1. A patient controlled utilization receptacle comprising:
a touch control switch which is responsive to changes in capacitance,
a control wire connected to an input of the touch control switch and which is of a sufficient length to be positionable adjacent the patient to be touched by the patient;
a relay having input contacts electrically connected to an output of the touch control switch, and at least one contact plate movable between an on position in which the contact plate connects a pair of load contacts and an off position in which the contact plate is disengaged from the pair of load contacts; and
a patient-controlled utilization outlet into which equipment, to be activated, can be plugged; the patient-controlled utilization outlet being electrically connected to the load contacts of the rely switch;
said touch control switch, said control wire, and said input to said relay defining a control circuit of said patient controlled utilization receptacle, and said contact plate and load contacts of said relay and said patient-controlled utilization outlet defining a switched circuit of said patient controlled utilization receptacle;
whereby contact with the control wire by the patient will activate the touch control switch, causing the relay to move the contact plate from the off position to the on position, to thereby close the switched circuit to provide power to the patient-controlled utilization outlet.

2. The patient controlled utilization receptacle of claim 1 wherein the contact plate is moved from the “on” position to the “off” position to thereby open the switched circuit by a second activation of the touch control switch; such that the patient controls both activation and deactivation of equipment plugged into the patient-controlled utilization outlet.

3. The patient controlled utilization receptacle of claim 1 further including a by-pass circuit containing a by-pass outlet; such that said by-pass outlet is continuously powered and is not affected by operation of the touch control switch circuit.

4. The patient controlled utilization receptacle of claim 3 wherein said by-pass outlet and said patient-controlled utilization outlet are proximate each other on said patient controlled utilization receptacle, but are electrically isolated from each other.

5. The patent controlled utilization receptacle of claim 1 further including an indicator light in said switched circuit; said indicator light being activated when said switched circuit is closed to indicate that power is flowing to the patient-controlled utilization outlet.

6. The patient controlled utilization receptacle of claim 1 including a fuse or a current transformer circuit in the control wire or between the control wire and the touch control switch.

7. The patient controlled utilization receptacle of claim 1 wherein the circuits of the patient controlled utilization receptacle are contained within an enclosure; said control wire extending through a panel of said enclosure; whereby if said enclosure is made from metal, said control wire is electrically insulated from said control panel.

8. The patient controlled utilization receptacle of claim 1 wherein the load contacts of the relay are normally open contacts.

9. The patient controlled utilization receptacle of claim 5 wherein the relay is a double-pole double-through relay comprising first and second pairs of load contacts; said patient controlled utilization outlet being connected to the first pair of load contacts and the indicator light.
